(19) United States
(12) Patent Application Publication
(10) Pub. No.: US 2022/0008301 A1
(43) Pub. Date: Jan. 13, 2022

(54) COMPOSITIONS AND METHODS FOR REDUCING SILVER STAINING ON TEETH

(71) Applicant: D-Stain, LLC, San Francisco, CA (US)

(72) Inventors: Jong Seto, San Francisco, CA (US); Jeremy Horst Keeper, San Francisco, CA (US)

(21) Appl. No.: 17/362,653

(22) Filed: Jun. 29, 2021

Related U.S. Application Data
(60) Provisional application No. 63/045,748, filed on Jun. 29, 2020.

(51) Int. Cl. A61K 8/22; A61K 8/20; A61K 8/24; A61Q 11/00; A61C 19/06; A61C 17/00

(52) U.S. Cl. CPC A61K 8/22; A61K 8/20; A61K 8/24; A61K 2800/87; A61C 19/066; A61C 17/00; A61K 2800/884; A61Q 11/00

(57) ABSTRACT

Methods and compositions for reducing silver stains on teeth treated using silver diamine fluoride are provided. The methods for removing stains comprise the steps of etching a stained area, the application of sodium hypochlorite, the application of hydrogen peroxide and the application of a remineralization solution to the stained area.

### CROSS REFERENCE TO RELATED APPLICATION

The present application claims priority to and the benefit of U.S. provisional patent application No. 63/045,748 filed on Jun. 29, 2020, which is incorporated by reference in its entirety for any purpose.

### FIELD

The present disclosure relates to compositions and methods for reducing silver staining on teeth.

### BACKGROUND

Silver diamine fluoride (SDF) has been used to treat dental caries (tooth decay) and has been shown to prevent the formation of new caries lesions (Slayton et al., 2018. Evidence-based clinical practice guideline on nonrestorative treatments for carious lesions: A report from the American Dental Association. J Am Dent Assoc. 149:837; Seifo et al., 2019. Silver diamine fluoride for managing carious lesions: an umbrella review. BMC Oral Health. 19:145, the entirety of which are herein incorporated by reference for any purpose). However, treating and preventing carious lesions (cavities) with SDF or silver nitrate and fluoride varnish (SN+FV) can result in a dark stain to treated tooth surfaces. These stains may be a result of treating areas of teeth that are either demineralized or hypomineralized. General societal expectations favor white teeth, so there is a potential for social stigma from such staining on teeth, leading to underutilization of these otherwise effective and beneficial treatment regimens. Traditional processes to remove the stain require excavation with a dental drill, which is counter to the value of non-invasive cavity treatment with SDF. Some SDF-stains can be covered with certain opaque dental materials, but for esthetic areas (visible areas toward the front of the mouth) this requires very high skill levels, and the materials often chip off.

Currently available teeth whitening compositions are generally ineffective for treatment of silver staining from SDF treatment. Moreover, various commercially available teeth whitening compositions can cause significant tooth sensitization in patients. Such tooth sensitivity may be the result of the movement of fluid through the dentinal tubules to the nerve endings due to the presence of glycerin, propylene glycol and polyethylene glycol in these compositions.

The use of teeth whitening compositions can also cause a number of other adverse events aside from sensitivity. Such adverse events include leaching of calcium from the enamel layer, penetration of the intact enamel and dentin by the bleaching agents, and dilution of the bleaching compositions with a person's saliva leading to leaching from the dental tray and ingestion by the user.

Thus, new methods for reducing or removing SDF stains from teeth using non-invasive means are desirable.

### SUMMARY

In various embodiments of the present disclosure, methods of treating a silver stain on a tooth are provided. In aspects of the present disclosure, a method of reducing or removing a stain on a tooth is provided that can comprise the following steps: cleaning a stained area of the tooth using an abrasive tool, isolating the tooth and applying petroleum jelly around the gingival margin, etching the stained area of the tooth with a phosphoric acid gel, rinsing the tooth, desiccating the tooth with air, applying a sodium hypochlorite solution to the stained area, applying hydrogen-peroxide to the stained area, rinsing the tooth, applying a remineralization solution to the stained area, and rinsing the tooth.

In various embodiments, steps of the etching, desiccating, sodium hypochlorite and hydrogen peroxide steps may be repeated. In some embodiments, various steps may be repeated sequentially.

In various embodiments, the phosphoric acid gel or phosphoric acid solution has a phosphoric acid concentration of at least about 38%. In various embodiments, the sodium hypochlorite solution has a sodium hypochlorite concentration of at least about 5%. In various embodiments, the sodium hypochlorite solution has a sodium hypochlorite concentration of at least about 8%. In various embodiments, the sodium hypochlorite solution has a sodium hypochlorite concentration of at least about 10% to about 15%.

In various embodiments, a method effective to achieve a reduction in silver staining may be performed in less than about 10 minutes. In various embodiments, a method effective to achieve a reduction in staining may be performed in less than about 5 minutes. In various embodiments, a method is effective for treatment of a silver stain located on the surface of the tooth. In various embodiments, a method is effective for treatment of a silver stain located within about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, or about 50 μm of the surface of the tooth.

In aspects of the disclosure, a method of reducing a silver stain on a tooth comprises cleaning the stained area of the tooth using an abrasive tool, isolating the tooth and applying petroleum jelly around the gingival margin, etching the stained area of the tooth with a phosphoric acid gel, rinsing the tooth, desiccating the tooth with air, applying a sodium hypochlorite solution to the stained area, applying hydrogen-peroxide to the stained area, rinsing the tooth, and applying a remineralization solution to the stained area.

In aspects of the disclosure, a kit for the treatment of a stained tooth that includes an abrasive cleaning tool, a tool to isolate the stained tooth, petroleum jelly, a tool to etch the stained area, a composition comprising sodium hypochlorite and a microbrush tool to apply the sodium hypochlorite to the stained area, a composition comprising hydrogen peroxide, a composition comprising a remineralization solution, and instructions for use, is provided. In various embodiments, the sodium hypochlorite solution has a sodium hypochlorite concentration of at least about 5%. In various embodiments, the hydrogen peroxide has a concentration of at least about 3%. In various embodiments, the dental etch comprises a phosphoric acid solution having a concentration of about 38%. In various embodiments, the remineralization solution comprises a calcium phosphate solution with a concentration of about 10 mM calcium phosphate.

In aspects of the disclosure, a method for removing a silver stain from a tooth is provided, the method comprising applying a sodium hypochlorite solution to a silver stained area comprising a visible silver oxide material on a surface of a tooth treated with silver diamine fluoride, wherein the application of the sodium hypochlorite solution is effective to remove a substantial amount of the visible silver oxide material from the surface of the tooth. In various embodiments, the sodium hypochlorite solution has a sodium hypochlorite concentration of at least about 5%.

In aspects of the disclosure, a method for removing silver stain from a tooth comprising, applying sodium hypochlorite to a stained area of a tooth treated with silver diamine fluoride to produce radical oxygen and chlorine, wherein the radical oxygen hydrolyzes proteins that are attached to the silver stain and the chlorine precipitates white silver chloride, is provided.

In aspects of the disclosure, a method for removing silver stain from a tooth comprising, applying a phosphoric acid solution to a silver stained area comprising a visible silver oxide material on a surface of a tooth treated with silver diamine fluoride, rinsing the silver stained area of the tooth, drying the silver stained area of the tooth, and applying a sodium hypochlorite solution to the silver stained area of the tooth, wherein the silver stain is removed from the tooth is provided.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures.

FIG. 1 consists of photographs from a time course showing the effects of a treatment method in accordance with various embodiments of the present disclosure on an SDF silver stained tooth.

FIG. 2 consists of photographs of a patient's teeth before and after treatment using a method in accordance with various embodiments of the present disclosure.

FIG. 3 consists of photographs of a patient's teeth before and after treatment using a method in accordance with various embodiments of the present disclosure.

FIG. 4 consists of photographs of a patient's teeth before and after treatment using a method in accordance with various embodiments of the present disclosure.

### DETAILED DESCRIPTION

The mechanism of caries arrest by silver diamine fluoride (SDF) has previously been investigated and it was discovered that dark staining caused by SDF treatment is comprised of various silver oxides that are bound to organic materials in the tooth. Without wishing to be bound by theory, the following effects of SDF treatment have been observed and/or hypothesized to occur in SDF treated areas of a tooth. Treatment with SDF is believed to produce deposits of a layer of silver and/or silver-conjugated protein compounds that are resistant to cariogenic processes, including, for example, acids produced by cariogenic bacteria on the treated area of the tooth. Calcium fluoride may be produced that can act as a reservoir for fluoride by neutralizing the imbalance between demineralization and remineralization. Hydroxyapatite and fluorapatite may form on the treated tooth surface together with silver chloride and metallic silver. As a result, a treated tooth surface may have an increased hardness as compared to the untreated tooth. The caries formation process is arrested, and the decayed area is often stained brown or black due to oxidation of the silver present in the deposited silver and silver-conjugated organic material, presumably to a silver oxide form. Further details of the SDF treatment mechanism and effects are described in Horst et al. 2016, UCSF Protocol for Caries Arrest Using Silver Diamine Fluoride: Rationale, Indications, and Consent, J. Calif. Dent. Assoc., 44(1): 16-28; Mei et al., Formation of Fluorohydroxyapatite with Silver Diamine Fluoride, J. Dent. Res., 96(10): 1122-1128; Zhao et al. 2018. Mechanisms of Silver Diamine Fluoride on Arresting Caries: A Literature Review. Int. Dent. J., 68: 67-76; the entireties of which foregoing disclosures are hereby incorporated by reference for any purpose.

As described in the present disclosure, it has been discovered that SDF staining of treated teeth can be reduced or removed by chemically degrading at least a portion of the exposed silver-conjugated organic material and/or converting at least a portion of the silver deposit that results from SDF treatment of a cavity to a non-dark form. In various embodiments, sodium hypochlorite may be used to reduce an SDF silver deposit on a treated tooth. The SDF silver deposit can be chemically reduced to reduce (decrease) or reverse its dark color. In various embodiments of the present disclosure, radical oxygen from hypochlorite may hydrolyze protein or other organic matter components of a silver-conjugated organic matter as well as solubilize free silver into solution that is present in a stained area of an SDF treated tooth. Chlorine from sodium hypochlorite treatment can precipitate silver chloride, which may result in whitening of the stained area. Methods of treatment using sodium hypochlorite to reduce or remove silver staining of SDF treated teeth are provided.

The terminology used herein is for the purpose of describing particular embodiments only and is not intended to be limiting. It must be noted that, as used in this specification and the appended claims, the singular forms “a,” “an,” and “the” include plural referents unless the context clearly dictates otherwise. Thus, for example, reference to “an oxidizing agent” includes not only a single oxidizing agent but also a combination or mixture of two or more different oxidizing agents, and the like.

The compositions and methods of the present disclosure may reduce or remove tooth stains without subjecting the person to the adverse events associated with other tooth whitening compositions, such as tooth sensitivity.

The methods described herein comprise non-invasive techniques to reduce or remove SDF stains on the surface of treated teeth. In various embodiments of the present disclosure, reduction or removal of SDF silver deposits from a treated tooth is achieved by substantially chemical means, not by mechanical removal of the SDF silver deposit. In embodiments, a SDF stain may be present on the surface of a tooth in the outer surface of a cavity and/or may be present beneath the surface of a cavity, for example, within the body of the tooth where the cavity is located. In embodiments, a method of treatment may remove a SDF stain from a tooth located within about 1 μm, or about 2 μm, or about 3 μm, or about 4 μm, or about 5 μm, or about 6 μm, or about 7 μm, or about 8 μm, or about 9 μm, or about 10 μm, or about 11 μm, or about 12 μm, or about 13 μm, or about 14 μm, or about 15 μm, or about 16 μm, or about 17 μm, or about 18 μm, or about 19 μm, or about 20 μm, or about 25 μm, or about 30 μm, or about 35 μm, or about 40 μm, or about 45 μm, or about 50 μm within the tooth surface.

The methods for reducing or removing a silver stain on a tooth may comprise a step of cleaning the stained area of a tooth with an abrasive tool. In aspects of the disclosure, an abrasive tool includes, but is not limited to, an abrasive strip, a pumice, a brush, for example, a contra-angle brush, or any dental instrument to which an abrasive tool may be added. An abrasive agent, for example an abrasive paste or gel may be added to an abrasive tool or any dental instrument to create an abrasive area to clean the stained area of the tooth surface. In accordance with various embodiments of the methods described herein, a cleaning step using an abrasive tool does not produce any substantial removal of SDF silver deposits within the tooth or the corresponding staining of a stained area.

In embodiments, a method may be effective to treat silver staining caused by use of a dental light cure or chemical precipitant that may have been used as part of patient treatment using SDF and may have caused precipitation of silver deposits. Such treatment may produce an extrinsic stain or secondary stain on the external surface of a tooth due to the release or precipitation of silver as secondary silver deposits. In embodiments, a secondary stain may occur on the same tooth previously treated or on a different tooth. In embodiments, a secondary stain may be removed using the methods as described herein.

In aspects of the disclosure, the stained area of a tooth can be isolated using various methods, including, but not limited to, cotton padding, a dental dam, an Isolite (Zyris, Santa Barbara. Calif.) or similar system, or any combination thereof. Isolation of the operative site from the rest of the mouth may be useful to prevent saliva from interfering with the dental work, or to prevent instruments and materials from being inhaled, swallowed or injuring oral tissues. In embodiments, the stained area of a tooth may be dried during the treatment process using any method and/or material capable of drying the area of the tooth to be treated. In embodiments, the stained area of a tooth may be dried during the treatment process using cotton padding, compressed air, or the like.

In aspects of the disclosure, a gingival barrier may be applied around the gingival margin of the tooth to be treated to create a physical short-term barrier. The gingival barrier may be removed after the treatment step has been completed. In embodiments, a petroleum jelly product can be applied as a gingival barrier around the gingival margin of the tooth to be treated. The petroleum jelly product may be any commercially available petroleum jelly, for example, Vaseline® (Unilever). The petroleum jelly protects the gum line around the tooth being treated. The petroleum jelly may be applied using an applicator, for example, a cotton applicator. The petroleum jelly may be reapplied at any step or steps during the treatment process as applicable. In embodiments, other gingival dam products such as OpalDam (UltraDent, South Jordan, Utah) may be applied as a gingival barrier around the gingival margin of the tooth to be treated. OpalDam is a light-cured resin barrier that creates a passively adhesive (sealing) methacrylate-based resin barrier and may be used for isolating tissue adjacent to a tooth being treated using the methods herein. This step in the treatment process may be repeated one or more times as applicable.

In aspects of the disclosure, the stained area of the tooth is etched using an acid etchant including, but not limited to, phosphoric acid gel, hydrochloric acid gel, or the like. A phosphoric acid gel acid etchant can be used at a concentration of from about 20% to about 50%. In various embodiments, a phosphoric acid gel can be used at a concentration of at least about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50%. In embodiments, a phosphoric acid gel is used at a concentration of about 34 to 40%. In some embodiments, a phosphoric acid gel is used at a concentration of about 38%. A hydrochloric acid gel acid etchant can be used at a concentration of from about 5% to about 38%. In various embodiments, a hydrochloric acid gel can be used at a concentration of at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 32%, 34%, 36%, or about 38%. In embodiments, a hydrochloric acid gel is used at a concentration of about 10 to about 20%. In some embodiments, a hydrochloric acid gel is used at a concentration of about 15%. An acid etchant may be used to remove surface fluoroapatite/hydroxyapatite that may have formed over the SDF-treated area. Any suitable acid etchant at any suitable acid concentration may be used in accordance with various embodiments. This step in the treatment process may be repeated one or more times as applicable.

In aspects of the disclosure, the stained area may be desiccated using air. This step may be performed at any step or steps during the treatment process as applicable, including, for example, as an intermediate step between applications of different solutions to the treated area. This step in the treatment process may be repeated one or more times as applicable.

In aspects of the disclosure, sodium hypochlorite may be applied to the stained area. The sodium hypochlorite can be applied as a gel or a liquid solution. The sodium hypochlorite can be used at a concentration of at least about 1% to 20%. In some embodiments, the sodium hypochlorite can be used at a concentration of at least about 1%, 2%, 3%, 4%, 5%, 6%, 7%, 8%, 9%, 10%, 11%, 12%, 13%, 14%, 15%, 16%, 17%, 18%, 19%, or about 20%. In some embodiments, the sodium hypochlorite is used at a concentration of about 8%. The sodium hypochlorite may be applied using an applicator, for example, a microbrush. This step in the treatment process may be repeated one or more times as applicable.

In aspects of the disclosure, the application of sodium hypochlorite to a stained area causes a chemical reaction that can remove a dark stain on a tooth caused by SDF treatment. Without wishing to be bound by theory, the hypochlorite may produce radical oxygen that can hydrolyze the proteins that are conjugated with or otherwise bound to the silver in the SDF stained area. The chlorine can precipitate as silver chloride, which may have a white color. In various embodiments of the present disclosure, the methods described herein may produce one or both of the foregoing reactions, which can result in the SDF silver stain being reduced or removed from the treated tooth. This aspect as part of the treatment methods set forth herein may result in the whitening of an SDF stained tooth.

In embodiments, other proteolytic agents or enzymes, for example papain, may be used in the methods to hydrolyze the proteins that are conjugated with or otherwise bound to the silver in the SDF stained area. In embodiments, the hydrolyzation of a silver-conjugated protein during treatment may cause the silver to be released. The silver may be solubilized into solution that is present in the tooth area during the treatment process. Additional embodiments include use of chelators to disrupt free silver conjugates from proteins and tooth surface. In embodiments, a gel comprised of papain and chloramine (for example, Papacarie® gel (Anvisa, Brazil)) may be used in the methods to hydrolyze the proteins conjugated or otherwise bound to silver in the SDF stained area. Without wishing to be bound by theory, use of a proteolytic agent in connection with the methods disclosed herein may enhance the efficacy of the reduction in staining.

In aspects of the disclosure, the stained area being treated can be kept moist using the sodium hypochlorite solution for a period of time. The period of time may be referred to as the treatment period. In various embodiments, the treatment period may be in a range of from about 10 seconds to about 30 minutes. In various embodiments, the treatment period may be sufficient to achieve a substantial reduction or removal of the SDF silver stain. In various embodiments, the treatment period may be about 30 s, or about 45 s, or about 60 s, or about 90 s, or about 2 min, or about 3 min, or about 4 min, or about 5 min, or about 6 min, or about 7 min, or about 8 min, or about 9 min. or about 10 min, or about 15 min. or about 20 min, or about 25 min. or about 30 min, or any time period of intermediate duration within the foregoing range. The sodium hypochlorite solution application step may be performed at any step or steps during the treatment process as applicable. This step in the treatment process may be repeated one or more times as applicable.

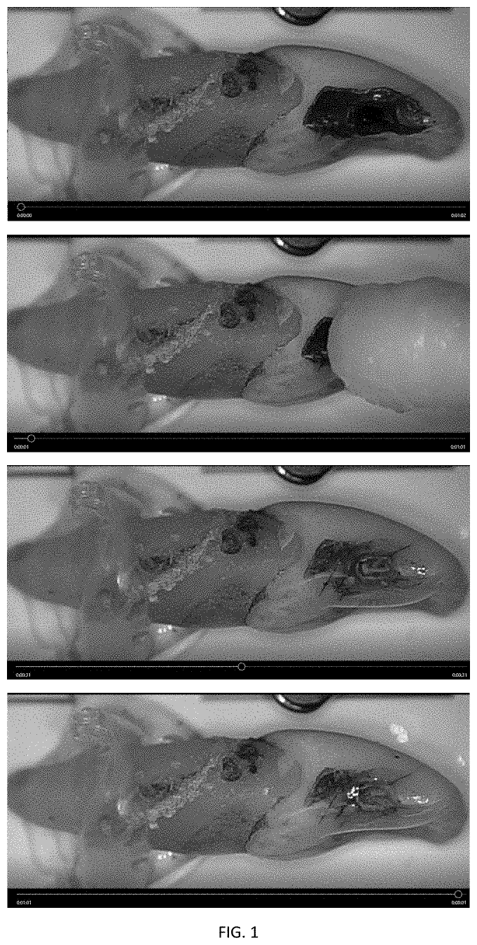

With reference now to FIG. 1, the effects of treatment of an SDF stained area of an extracted tooth with an 8% sodium hypochlorite solution in accordance with various embodiments of the present disclosure is illustrated. The images illustrate the effects of treatment of the SDF stained area with a sodium hypochlorite solution over the course of approximately 60 seconds, the images being captured from a video recording of the treatment period. The top photograph shows the tooth prior to application of a sodium hypochlorite solution. The second photograph shows initiation of the sodium hypochlorite solution application to the extracted tooth. The third photograph shows the condition of the treated tooth after about 30 seconds of sodium hypochlorite solution application to the tooth, and the fourth photograph shows the treated tooth after about 60 seconds of sodium hypochlorite solution application to the tooth. As can be seen from the photographs in FIG. 1, application of a sodium hypochlorite solution to the SDF stained tooth substantially reduces the area and intensity (darkness) of the SDF silver staining within the 60 second treatment period.

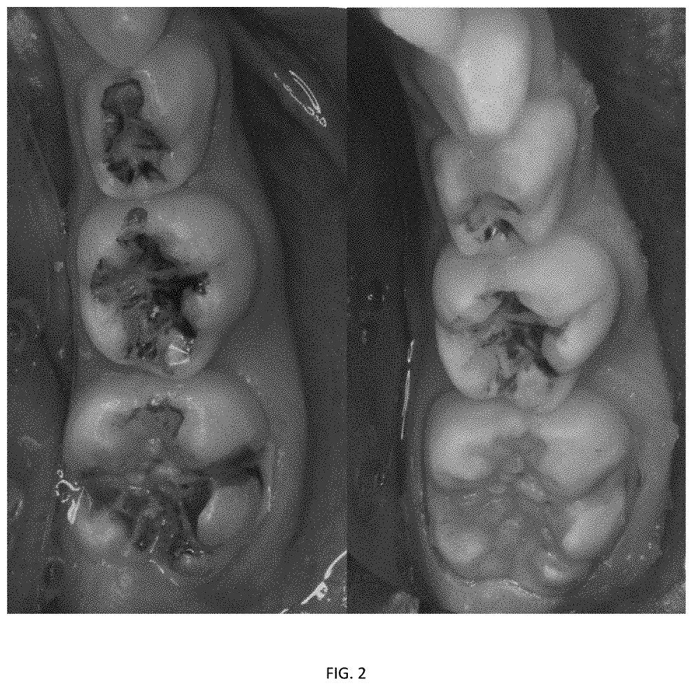

With reference now to FIG. 2, the effects of treatment of teeth with SDF stained areas with an 8% sodium hypochlorite solution using a method in accordance with various embodiments of the present disclosure is illustrated. The photograph on the left shows the teeth prior to application of a sodium hypochlorite solution. The photograph on the right shows the treated teeth after about 100 seconds of sodium hypochlorite solution application to the teeth. As can be seen from the photographs in FIG. 2, application of a sodium hypochlorite solution to the SDF stained teeth using methods disclosed herein substantially reduces the area and intensity (darkness) of the SDF silver staining within a 100 second treatment period.

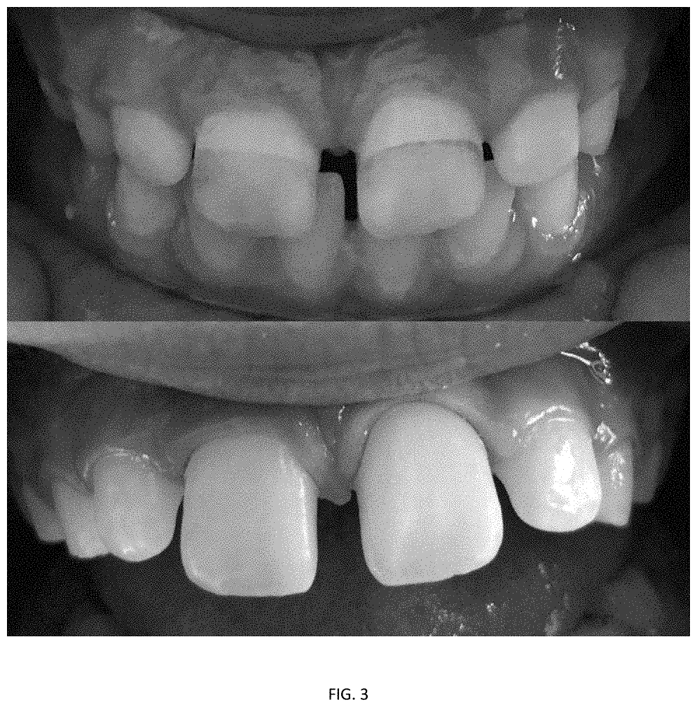

With reference now to FIG. 3, the effects of treatment of teeth with SDF stained areas with an 8% sodium hypochlorite solution using a method in accordance with various embodiments of the present disclosure is illustrated. The upper photograph shows the teeth prior to application of a sodium hypochlorite solution. The lower photograph shows the treated teeth after about 60 seconds of sodium hypochlorite solution application to the teeth. As can be seen from the photographs in FIG. 3, application of a sodium hypochlorite solution to the SDF stained teeth using methods disclosed herein substantially reduces the area and intensity (darkness) of the SDF silver staining within a 60 second treatment period.

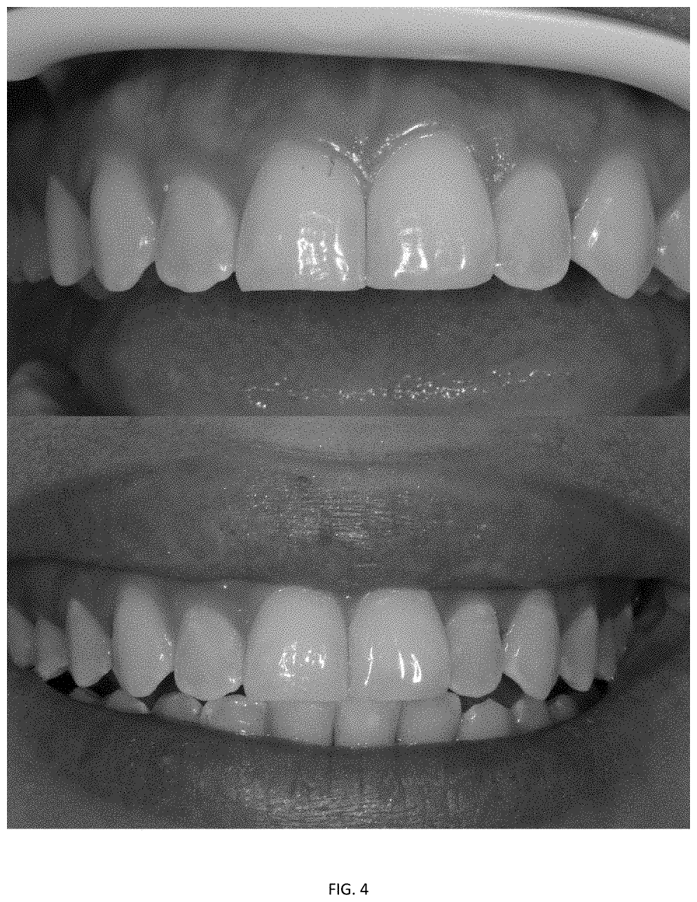

With reference now to FIG. 4, the effects of treatment of teeth with SDF stained areas with an 8% sodium hypochlorite solution using a method in accordance with various embodiments of the present disclosure is illustrated. The upper photograph shows the teeth prior to application of a sodium hypochlorite solution. The lower photograph shows the treated teeth after about 60 seconds of sodium hypochlorite solution application to the teeth. As can be seen from the photographs in FIG. 4, application of a sodium hypochlorite solution to the SDF stained teeth using methods disclosed herein substantially reduces the area and intensity (darkness) of the SDF silver staining within a 60 second treatment period.

In aspects of the disclosure, an oxidizing agent, for example, hydrogen peroxide, may be applied to the stained area being treated. The hydrogen peroxide may be applied using an applicator, for example, a microbrush. The hydrogen peroxide may be applied in small quantities. In embodiments, hydrogen peroxide may be applied at a concentration of at least about 0.5%, 1%, 2%, 3%, 4%, 5%, 6%, 7%, 8%, 9%, or 10%. In embodiments, hydrogen peroxide may be applied at a concentration of at least about 3%. Any excess of hydrogen peroxide can be removed from the applicator prior to application to the stained area being treated. A suction apparatus may be used to remove any gases emitted from the hydrogen peroxide. This step in the treatment process may be repeated one or more times as applicable.

In aspects of the disclosure, the stained area being treated can be rinsed with an appropriate solution. In embodiments, the stained area may be rinsed with water or a solution comprising witch hazel. This step may be performed at any step or steps during the treatment process as applicable. This step in the treatment process may be repeated one or more times as applicable. The rinse step in the treatment process may be performed for any amount of time sufficient to have the desired effect. In embodiments, a rinse step may be performed for at least about 5, 10, 15, 20, 25 or 30 seconds. In embodiments, the rinsing step may prevent the release or precipitation of silver from forming a secondary silver deposit or secondary stain.

In aspects of the disclosure, a remineralization solution may be applied to the stained area using an applicator. In embodiments, the remineralization solution includes calcium phosphate. The calcium phosphate may be at a concentration of at least about 10 mM. The remineralization solution is applied to the stained area for at least about five seconds. A rinsing step may be performed using an appropriate solution, for example, water. This step may be performed at any step or steps during the treatment process as applicable. This step in the treatment process may be repeated one or more times as applicable.

In aspects of the disclosure, the method of treatment steps may be repeated. In embodiments, one or more steps of the treatment method may be repeated one or more times. In embodiments, one or more steps of the treatment method may be repeated once, twice, three or more times. The treatment steps may be repeated until the staining of the stained area is reduced or removed.

In aspects of the disclosure, a method of treatment may be completed in a short time period, such as from less than about 60 minutes to less than about five minutes. In various embodiments, the method steps are completed in about five minutes to about 10 minutes.

In aspects of the disclosure, the method steps are completed in a specific order. In embodiments, the steps of etching with phosphoric acid, application of sodium hypochlorite, and application of hydrogen peroxide are performed sequentially. The foregoing steps may be repeated in this sequence one or more times.

In aspects of the disclosure, the described methods may be effective to reduce or remove an SDF silver stain on a tooth. In embodiments, the methods of the present disclosure can reduce or remove a stain on a tooth such that a desired result is achieved. A reduction or removal of a stain is an improvement, modification, change or alteration in the appearance of the stain. The reduction or removal of the stained area on a tooth is significant to achieve a desired result, for example, whiter teeth. In embodiments of the disclosure, the stained area and/or stain intensity (i.e., darkness or color) is reduced such that the stain is less prominent and noticeable. In various embodiments, a stain may be removed completely.

In various embodiments, treatment of an SDF stained tooth may be effective to reduce the staining of a treated tooth in an objectively quantifiable manner. The shade or color of an SDF stained tooth may be measured before and after treatment in accordance with various methods described herein. In various embodiments, a treatment may be effective to produce a measurable lightening of the treated tooth by visual comparison to a shade scale or as measured using an instrument such as a spectrophotometer or image analysis system. In various embodiments, a scale such as the VITA Classical Shade Guide. CIELAB (color space specified by the international Commission on Illumination) or similar scale may be used, or simply grayscale. In embodiments, a spectrophotometric system such as a VITA Easyshade Spectrophotometer. SpectroShade spectrophotometer or similar spectrophotometer may be used to measure tooth shading. In embodiments, the shade or color of an SDF stained tooth may lighten from a brownish hue, yellowish hue, greyish hue or whitish hue to a lighter shade or color after treatment. In embodiments, the shade or color of an SDF stained tooth may lighten to a color having similar chroma or a similar color. In embodiments, the shade or color of an SDF stained tooth may show a subtle change in shade or color or may show a visibly noticeable change in shade or color. Tooth shading may be determined based on a topographical color mapping of a whole tooth. A digital camera may be used to obtain a digital image of a tooth, and the image used to objectively measure the tooth color, for example, with reference to an external standard or another objective reference (such as a VITA scale or CIELAB color space). Any system or method facilitating objective and/or quantitative measurement of stain lightening may be used in accordance with various embodiments of the present disclosure.

In various embodiments, a method of treating an SDF stained tooth may be effective to achieve at least 1 shade, or at least 2 shades, or at least 3 shades of stain lightening for a treated tooth or a treated area of a tooth. In various embodiments, a method of treating an SDF stained tooth may be effective to reduce the area and/or degree of staining by at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97%, or at least about 98%, or at least about 99%, or at least about 100%.

In aspects of the invention, a method of treating an SDF stained tooth may be effective to reduce or eliminate the presence of microorganisms, including oral pathogens. Treatment of an SDF stained tooth using the methods presented hereof may reduce or eliminate the presence of oral microorganisms. Without wishing to be bound by theory, in embodiments, the presence of microorganisms may be eliminated or reduced by the process of applying a sodium hypochlorite solution and applying hydrogen-peroxide to the stained area of a tooth prior to applying a remineralization solution to the stained area of a tooth. The reduction or elimination of one or more microorganisms may reduce future or further dental caries. A reduction in the presence of one or more microorganisms may be measured using any of a variety of assays including, but not limited to, direct cell counting (for example, microscopic enumeration, electronic enumeration), culture-based methods, adenosine triphosphate bioluminescence assays, polymerase chain reaction based assays, or any other suitable assay. In various embodiments, a patient's entire mouth may be sampled, or specific sites within the oral cavity of a patient may be sampled, such as a site adjacent to a SDF stained tooth to be treated using the methods and compositions of the present disclosure. Sampling of a patient's mouth to determine the presence or abundance of microorganisms may be performed prior to treatment of a patient to reduce SDF staining in accordance with embodiments of the present disclosure, and following such treatment to determine the effect of the treatment on the presence of microorganisms. The methods of treatment disclosed herein may result in the elimination of one or more microorganisms such that the presence or abundance of bacteria is reduced. The presence or abundance of bacteria may be determined to be reduced for the whole mouth or for a specific site.

### EXAMPLES

### Example 1

Cavities on extracted teeth were treated with SDF, the stain was allowed to set in for about twenty-four hours. The stain was treated with sodium hypochlorite and the stain was substantially reduced or removed. A number of different combinations and sequences were tested to catalyze or expedite the process to a clinically practical treatment period target time of five minutes.

### Example 2

Compositions with sodium hypochlorite at concentrations of 15% (industrial) and 8.25%, 5%, 1.67%, 0.56% and 0.18% (household) were prepared and tested. Compositions with sodium hypochlorite were prepared as gel or liquid compositions. Higher concentrations of sodium hypochlorite produced faster results. Compositions with about 5-15% sodium hypochlorite produced similar results.

The following compositions and methods were tested:

A. 38% phosphoric acid (dental etch), to remove surface fluoroapatite/hydroxyapatite that may have formed over the SDF-treated area. This step was demonstrated to help when performed before the addition of sodium hypochlorite.

B. Dental polish with pumice with or without glycerine (for example, a prophy) to mechanically remove the stain. This step was not effective.

C. Rinse witch hazel instead of water. This step demonstrated a minor improvement in decreasing the stain.

D. SDS-based detergents. The application of these detergents demonstrated no perceived benefit.

E. 3% hydrogen peroxide. The application of hydrogen peroxide created a bubbling effect that provided strong visual feedback upon application, but did not demonstrate any perceived benefit.

F. Potassium iodide. The application of potassium iodide demonstrated no perceived benefit.

G. Water with or without calcium phosphate (toner). The application of water demonstrated no perceived benefit.

### Example 3

Combinatory sequencing:

Each material was tried in combination with application of a sodium hypochlorite solution in various sequences based on the rationale of use. A material that demonstrated a benefit was included in the method steps based on its contribution towards substantially reducing or removing all stain and decreasing the time to remove the stain.

The combination of steps including, but not limited to, etch, bleach, and witch hazel were demonstrated to be effective. The step of hydrogen peroxide application may optionally be included for various functional and aesthetic reasons, including that the bubbling effect may enhance the user experience and provide a perceived value to the user.

The microstructural changes after the removal of a silver stain using the methods herein have been assessed. It has been discovered that a charcoal-like surface scab of visible silver staining resulting from SDF treatment, which has been characterized as about 10 μm thick and comprised of mixture of silver oxide organic conjugates, is substantially reduced or removed using the methods provided. Metallic silver microwires produced by SDF treatment remain within the microcavitations and dentinal tubules of the treated tooth after SDF silver stain treatment using the methods provided in the present disclosure. The microwires may contribute to the hardening of a cavity, to desensitization (treating sensitivity), and to resistance to the recurrence of the cavity.

### Example 4

The following steps were performed:

1. Clean: the stain area was cleaned using a contra-angle brush, followed with a pumice, and the area rinsed.

2. Isolate: the stain area was isolated using cotton and a dam. Petroleum jelly (for example. Vaseline) was placed around the gingival margins with a cotton applicator around the tooth to be treated. A bulk of petroleum jelly can be used.

3. The area was etched for about 30 seconds with 38% phosphoric acid gel and rinsed for about 15 seconds.

4. The area was desiccated with air. Petroleum jelly may be reapplied if the prior application has been removed by the etch/rinse/dry steps.

5. An 8% sodium hypochlorite solution was applied using a microbrush. The stained areas were kept moist for at least about one minute. Cloudy solution was removed using a dry cotton applicator and fresh sodium hypochlorite solution reapplied using the microbrush. No rinse was applied.

6. Sparing amounts of a hydrogen peroxide solution were applied with a microbrush to the areas where the sodium hypochlorite solution was applied. A bubbling effect was observed. A suction apparatus was used to remove gases emitted from the hydrogen peroxide bubbling. The hydrogen peroxide solution was applied for about one minute. The area was rinsed.

7. Steps 3-6 were repeated until a desired reduction of the stained areas was achieved.

8. A remineralization solution with 10 mM calcium phosphate was applied using a cotton applicator to the area where the sodium hypochlorite was placed. The area was left for five seconds. The area was rinsed.

The above steps were performed sequentially, and certain steps were repeated until the stained area was changed to white and the stain was removed. Successful silver stain removal in permanent teeth was achieved with the above method.

### Example 5

Silver stain removal was achieved using the method steps provided in Example 3. The application of sodium hypochlorite to a stained area of a tooth previously treated with silver diamine fluoride resulted in the production and release of radical oxygen and chlorine. The presence of radical oxygen was demonstrated to hydrolyze proteins that were attached to the silver stain on the tooth. The presence of the chlorine was demonstrated to precipitate as a white silver chloride. The combination of the protein hydrolysis and precipitation was shown to substantially reduce the area and darkness of the silver stain.

### Example 6

The effect on the silver stain on a tooth using the methods provided herewith was quantified. Six stained teeth were treated using the methods provided herewith. “S” and “T” represent baby teeth. To determine the percent change in the color of a treated tooth, the following formula was used to measure the percentage of color change.

Formula I: Δ=(destained−stained)/(white area−stained)

The difference in the 8-bit greyscale values was determined using the above formula to calculate the percentage of color change before and after treatment. The percentage color change was calculated for the worst area of the stained tooth and for the entire stain. For values that were calculated above 100% color change due to the whitest area of the tooth being the destained area, these numbers were flattened to a total of 100%.

TABLE 1

| Tooth | Δ Worst area | Δ Entire stain |
|---|---|---|
| 8 | 98% | 97% |
| 30 | 99% | 100% |
| S | 66% | 90% |
| T | 39% | 58% |
| 8 | 99% | 100% |
| 9 | 100% | 100% |

For each tooth, a difference of at least 58% for the entire stain was calculated. For the adult teeth, a difference of at least 98% was calculated for the worst area of the stained tooth and at least 97% for the entire stain. The data demonstrates a substantial color change in a stained tooth is achieved using the methods provided.

### Example 7

The clinical efficacy of treatment of teeth with SDF stained areas was demonstrated using the following method:

A 38% phosphoric acid etch solution was applied to an SDF stained area of a tooth for 15 seconds. The area was rinsed with water for 10 seconds and dabbed dry with cotton. An 8% sodium hypochlorite solution was applied to the SDF stained area as follows:

FIG. 2, 8% sodium hypochlorite was applied to the SDF stained tooth for 100 seconds.

FIG. 3, 8% sodium hypochlorite was applied to the SDF stained tooth for 60 seconds.

FIG. 4, 8% sodium hypochlorite was applied to the SDF stained tooth for 60 seconds.

The SDF stained area was rinsed with a witch hazel solution for 10 seconds and dabbed dry with cotton. A 3% hydrogen peroxide solution was applied for 50 seconds, rinsed with witch hazel for 10 seconds and rinsed with water.

The above process was repeated three times for the SDF stained teeth shown in FIG. 2, and once for the SDF stained teeth shown in FIG. 3 and FIG. 4.

### Example 8

The effects of treatment of teeth with SDF stained areas with an 8% sodium hypochlorite solution was demonstrated using the method in Example 7. The reduction in the SDF silver stain after treatment is shown in FIG. 2. The photograph on the left shows the teeth prior to the application of the 8% sodium hypochlorite solution. The photograph on the right shows the treated teeth after about 100 seconds of sodium hypochlorite solution application to the teeth. As can be seen from the photographs in FIG. 2, the application of an 8% sodium hypochlorite solution to the SDF stained teeth substantially reduced the area and intensity (darkness) of the SDF silver staining within a 100 second treatment period.

### Example 9

The effects of treatment of teeth with SDF stained areas with an 8% sodium hypochlorite solution was demonstrated using the method of Example 7. The reduction in the SDF silver stain after treatment is show in FIG. 3. The upper photograph shows the teeth prior to the application of an 8% sodium hypochlorite solution. The lower photograph shows the treated teeth after about 60 seconds of sodium hypochlorite solution application to the teeth. As can be seen from the photographs in FIG. 3, the application of an 8% sodium hypochlorite solution to the SDF stained teeth substantially reduced the area and intensity (darkness) of the SDF silver staining within a 60 second treatment period.

### Example 10

The effects of treatment of teeth with SDF stained areas with an 8% sodium hypochlorite solution was demonstrated using the method in Example 7. The reduction in the SDF silver stain after treatment is shown in FIG. 4. The upper photograph shows the teeth prior to the application of an 8% sodium hypochlorite solution. The lower photograph shows the treated teeth after about 60 seconds of sodium hypochlorite solution application to the teeth. As can be seen from the photographs in FIG. 4, the application of an 8% sodium hypochlorite solution to the SDF stained teeth substantially reduced the area and intensity (darkness) of the SDF silver staining within a 60 second treatment period.

It will be understood that the embodiments of the present disclosure can be modified in myriad ways other than those specifically discussed without departing from the scope of the disclosure. General variations to these embodiments may include different tooth whitening compositions and method steps of applying the compositions.

Those skilled in the art will readily recognize that even though selected preferred embodiments of the disclosure have been depicted and described, it will be understood that various changes and modifications can be made other than those specifically mentioned above without departing from the spirit and scope of the disclosure, which is defined by the claims that follow.

What is claimed is:

1. A method of treating a silver stain on a tooth comprising:
applying a sodium hypochlorite solution to a treated area of the tooth comprising the silver stain, wherein the silver stain has a first area and a first shade;
maintaining the sodium hypochlorite solution in contact with the treated area for a first time period to produce a treated tooth and a treated silver stain having a second area and a second shade;
wherein one of the second area is less than the first area and the second shade is lighter than the first shade.

2. The method of claim 1, wherein the method further comprises the following steps:
cleaning one of the treated area or the first area with an abrasive tool and etching one of the treated area or the first area with a phosphoric acid solution prior to applying the sodium hypochlorite solution;
applying a hydrogen peroxide solution to the treated tooth; and
applying a remineralization solution to the treated tooth.

3. The method of claim 2, wherein the phosphoric acid solution has a concentration of at least about 38%.

4. The method of claim 1, wherein the sodium hypochlorite solution has a concentration of at least about 5%.

5. The method of claim 1, wherein the sodium hypochlorite solution has a concentration of at least about 8%.

6. The method of claim 1, wherein the sodium hypochlorite solution has a concentration of at least about 10% to about 15%.

7. The method of claim 2, wherein the method is performed in less than about 10 minutes.

8. The method of claim 2, wherein the method is performed in less than about 5 minutes.

9. The method of claim 1, wherein the silver stain is located on the surface of the tooth.

10. The method of claim 7, wherein the silver stain is located within about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, or 50 μm of the surface of the tooth.

11. A kit for the treatment of a silver stained tooth comprising:
a single-use container holding a volume of a sodium hypochlorite solution; and
a single use applicator for the sodium hypochlorite solution.

12. The kit of claim 11, further comprising:
an abrasive cleaning tool;
a single-use container holding a volume of a dental etch solution;
a single-use container holding a volume of a hydrogen peroxide solution;
a single-use container holding a volume of a remineralization solution; and
instructions for use.

13. The kit of claim 12, wherein the sodium hypochlorite solution has a concentration of at least about 5%.

14. The kit of claim 12, wherein the hydrogen peroxide has a concentration of at least about 3%.

15. The kit of claim 12, wherein the dental etch comprises a phosphoric acid solution having a concentration of about 38%.

16. The kit of claim 12, wherein the remineralization solution comprises a calcium phosphate solution with a concentration of about 10 mM.

17. A method for removing silver stain from a tooth comprising;
applying a sodium hypochlorite solution to a stained area of a tooth treated with silver diamine fluoride;
wherein the stained area comprises a visible silver oxide material on a surface of the tooth;
wherein the applying the sodium hypochlorite solution is effective to produce radical oxygen and chlorine,
wherein the radical oxygen is effective to hydrolyze proteins that are attached to the silver stain and the chlorine is effective to precipitate white silver chloride, and
wherein the applying the sodium hypochlorite solution is effective to remove a substantial amount of the visible silver oxide material from the surface of the tooth.

18. The method of claim 17, further comprising additional steps of:
applying a phosphoric acid solution to the stained area;
rinsing the stained area; and
drying the stained area;
wherein the additional steps are performed prior to the applying the sodium hypochlorite solution to the stained area.
